(19) United States
(12) Patent Application Publication
(10) Pub. No.: US 2026/0345965 A1
(43) Pub. Date: Nov. 5, 2026

(54) INTRODUCER DEVICES, SYSTEMS, AND METHODS FOR USE WITH MEDICAL DEVICES

(71) Applicant: Boston Scientific Scimed, Inc., Maple Grove, MN (US)

(72) Inventors: Julie Gagnon, Providence, RI (US); Andrew James Schaubhut, Bolton, MA (US); Sofia Alejandra Cordero Villalobos, Heredia (CR)

(73) Assignee: Boston Scientific Scimed, Inc., Maple Grove, MN (US)

(21) Appl. No.: 19/664,015

(22) Filed: Apr. 30, 2026

Related U.S. Application Data
(60) Provisional application No. 63/798,261, filed on May 1, 2025.

(51) Int. Cl. A61M 25/09; A61B 1/00; A61B 1/018

(52) U.S. Cl. CPC A61M 25/09041; A61B 1/00128; A61B 1/018

(57) ABSTRACT

An introducer device for use with an accessory device and/or an endoscope. The introducer device may include a housing having a proximal end and a distal end, a port at the proximal end of the housing, a tube extending within the housing and in fluid communication with the port, a lumen extending through the housing, and a blade positioned within the lumen between the proximal end of the housing and the distal end of the housing. The accessory device may include an elongate tube configured to extend through the lumen of the introducer and the blade may be configured to cut through a wall of the elongate tube. The tube extending within the housing may extend into a guidewire lumen of the elongate tube and may be configured to receive a guidewire.

### RELATED APPLICATIONS

This application claims the benefit of U.S. Provisional Patent Application Serial Number 63/798,261, filed May. 1, 2025, which application is incorporated by reference herein in its entirety.

### TECHNICAL FIELD

The disclosure pertains to medical devices, and methods for manufacturing and use of these medical devices. More particularly, the present disclosure pertains to introducer devices, systems, and methods for accessing a body lumen.

### BACKGROUND

A wide variety of intracorporeal and extracorporeal medical devices and systems have been developed for medical use, for example, for endoscopic procedures. Some of these devices and systems include guidewires, introducers, catheters, catheter systems, endoscopes, ablation devices, tome devices, delivery systems, stents, anchors, and the like. These devices and systems are manufactured by any one of a variety of different manufacturing methods and may be used according to any one of a variety of methods. Of the known medical devices, systems, and methods, each has certain advantages and disadvantages. There is an ongoing need to provide alternative medical devices and systems as well as alternative methods for manufacturing and using medical devices and systems.

### SUMMARY

This disclosure provides design, material, manufacturing method, and use alternatives for medical devices and medical systems. In one example, a guidewire introducer may include a housing having a proximal end and a distal end, a port at the proximal end of the housing, a tube extending within the housing and in fluid communication with the port, a lumen extending through the housing, and a blade positioned within the lumen between the proximal end of the housing and the distal end of the housing.

Additionally or alternatively to any of the examples above, the lumen may be configured to receive an elongate tube of an accessory device and the tube extends into the lumen.

Additionally or alternatively to any of the examples above, the tube may be configured to be inserted into a guidewire lumen of an elongate tube of an accessory device extending through the lumen.

Additionally or alternatively to any of the examples above, the lumen may have a distal opening at the distal end of the housing and a proximal opening through a sidewall of the housing between the distal end of the housing and the proximal end of the housing.

Additionally or alternatively to any of the examples above, the tube may extend into the lumen and the blade may extend between an outer surface of the tube and a surface defining the lumen.

Additionally or alternatively to any of the examples above, the guidewire introducer may include a tab located between the proximal end and the distal end of the housing.

Additionally or alternatively to any of the examples above, the tab may include a guidewire guide.

Additionally or alternatively to any of the examples above, the port may be configured to couple with a syringe.

Additionally or alternatively to any of the examples above, the housing may be configured to couple with a port of an endoscope.

Additionally or alternatively to any of the examples above, the tube may be a metallic tube and the blade may be metallic.

Additionally or alternatively to any of the examples above, the port may be a guidewire port and the guidewire introducer may further include a syringe port.

Additionally or alternatively to any of the examples above, the guidewire introducer further includes a hook for releasably securing a guidewire.

Additionally or alternatively to any of the examples above, the guidewire introducer is configured to position the hook above the port of the endoscope.

Additionally or alternatively to any of the examples above, the guidewire introducer further includes an extension portion that is configured to hold a guidewire relative to the guidewire introducer.

In another example, an accessory device for use with an endoscope may include a hub, an elongate tube having a proximal end at the hub, a distal end, and one or more lumens extending through the elongate tube, and a guidewire introducer coupled with the elongate tube, the guidewire introducer including a housing having a proximal end and a distal end, a tube extending within the housing into a first lumen of the one or more lumens, a lumen extending through the housing with the elongate tube extending through the lumen, and a blade extending through a wall of the elongate tube defining the first lumen.

Additionally or alternatively to any of the examples above, the tube may be positioned in the first lumen and may be configured to receive a guidewire extending through the first lumen.

Additionally or alternatively to any of the examples above, the elongate tube may extend through the distal end of the housing and a sidewall of the housing between the proximal end and the distal end of the housing.

Additionally or alternatively to any of the examples above, the guidewire introducer and the elongate tube may be configured to adjust longitudinally relative to one another.

Additionally or alternatively to any of the examples above, the blade may be configured to cut the wall of the elongate tube defining the first lumen as the guidewire introducer and the elongate tube adjust longitudinally relative to one another.

In another example, a method of using a guidewire introducer with an accessory device for insertion of a guidewire into an endoscope may include cutting a wall defining a guidewire lumen of an elongate tube of the accessory device with a blade of the guidewire introducer as the guidewire introducer is advanced over the elongate tube, inserting the guidewire through the guidewire introducer and the elongate tube to a target location, positioning the guidewire introducer proximate a port of the endoscope, the elongate tube extending through the port of the endoscope, and removing the elongate tube from the endoscope and the guidewire.

Additionally or alternatively to any of the examples above, the method may include removing the elongate tube from the guidewire introducer, wherein removing the elongate tube from the guidewire introducer includes cutting the elongate tube with the blade as the elongate tube is adjusted in a proximal direction.

Additionally or alternatively to any of the examples above, the method may further include removing the guidewire introducer from the guidewire.

Additionally or alternatively to any of the examples above, the method may further include extending a housing of the guidewire introducer through a seal at the port of the endoscope.

Additionally or alternatively to any of the examples above, the method may further include coupling a syringe with a port of a housing of the guidewire introducer and providing liquid to the guidewire lumen of the elongate tube.

These and other features and advantages of the present disclosure will be readily apparent from the following detailed description, the scope of the claimed invention being set out in the appended claims.

### BRIEF DESCRIPTION OF THE DRAWINGS

The accompanying drawings, which are incorporated in and constitute a part of this specification, illustrate various configurations and together with the description serve to explain the principles of the present disclosure.

FIG. 1 is a schematic view of an illustrative endoscope;

FIG. 2 is a schematic perspective view of an illustrative accessory device coupled with an illustrative introducer;

FIG. 3 is a schematic side view of an illustrative system comprising an endoscope, an accessory device, and an introducer;

FIG. 4 is a schematic perspective view of an illustrative introducer;

FIG. 5 is a schematic proximal end view of the illustrative introducer depicted in FIG. 4;

FIG. 6 is a schematic distal end view of the illustrative introducer depicted in FIG. 4;

FIG. 7 is a schematic cross-section view of the illustrative introducer depicted in FIG. 4, taken along line 7-7 in FIG. 6;

FIG. 8 is a schematic cross-section view of an illustrative introducer with an elongate tube of an accessory device inserted therethrough;

FIG. 9 is a schematic side view of an illustrative configuration of an introducer coupled with an adaptor or cap;

FIGS. 10A-10E are schematic side views of steps of a method of using a system comprising an endoscope, an accessory device, and an introducer;

FIG. 11 is schematic perspective view of an illustrative introducer;

FIG. 12 is a schematic cross-sectional view of the illustrative introducer of FIG. 11, with an elongate tube of an accessory device inserted therethrough; and

FIG. 13 is a schematic side view of a system including an endoscope, an accessory device, and the illustrative introducer of FIG. 12.

While the disclosure is amenable to various modifications and alternative forms, specifics thereof have been shown by way of example in the drawings and will be described in detail. It should be understood, however, that the intention is not to limit the invention to the particular configurations described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about”, in the context of numeric values, generally refers to a range of numbers that one of skill in the art would consider equivalent to the recited value (e.g., having the same function or result). In many instances, the term “about” may include numbers that are rounded to the nearest significant figure. Other uses of the term “about” (e.g., in a context other than numeric values) may be assumed to have their ordinary and customary definition(s), as understood from and consistent with the context of the specification, unless otherwise specified.

The recitation of numerical ranges by endpoints includes all numbers within that range, including the endpoints (e.g., 1 to 5 includes 1, 1.5, 2, 2.75, 3, 3.80, 4, and 5). Although some suitable dimensions, ranges, and/or values pertaining to various components, features, and/or specifications are disclosed, one of skill in the art, incited by the present disclosure, would understand desired dimensions, ranges, and/or values may deviate from those expressly disclosed.

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise. It is to be noted that in order to facilitate understanding, certain features of the disclosure may be described in the singular, even though those features may be plural or recurring within the disclosed configuration(s). Each instance of the features may include and/or be encompassed by the singular disclosure(s), unless expressly stated to the contrary. For simplicity and clarity purposes, not all elements of the disclosure are necessarily shown in each figure or discussed in detail below. However, it will be understood that the following discussion may apply equally to any and/or all of the components for which there are more than one, unless explicitly stated to the contrary. Additionally, not all instances of some elements or features may be shown in each figure for clarity.

It is noted that references in the specification to “a configuration”, “some configurations”, “other configurations”, etc., indicate that the configuration(s) described may include a particular feature, structure, or characteristic, but every configuration may not necessarily include the particular feature, structure, or characteristic. Moreover, such phrases are not necessarily referring to the same configuration. Further, when a particular feature, structure, or characteristic is described in connection with a configuration, the particular feature, structure, or characteristic may be effected in connection with other configurations, whether or not explicitly described, unless clearly stated to the contrary. That is, the various individual elements described below, even if not explicitly shown in a particular combination, are nevertheless contemplated as being combinable or arrangeable with each other to form other additional configurations or to complement and/or enrich the described configuration(s), as would be understood by one of ordinary skill in the art.

For the purpose of clarity, certain identifying numerical nomenclature (e.g., first, second, third, fourth, etc.) may be used throughout the description and/or claims to name and/or differentiate between various described and/or claimed features. The numerical nomenclature is not intended to be limiting and is illustrative only. In some configurations, alterations of and deviations from previously-used numerical nomenclature may be made in the interest of brevity and clarity. That is, a feature identified as a “first” element may later be referred to as a “second” element, a “third” element, etc. or may be omitted entirely, and/or a different feature may be referred to as the “first” element. The meaning and/or designation in each instance will be apparent to the skilled practitioner.

The detailed description is intended to illustrate but not limit the disclosure. Various elements described may be arranged in various combinations and configurations without departing from the scope of the disclosure. The detailed description illustrates example configurations of the disclosure.

Although endoscopes are referenced herein for illustration purposes, the disclosure encompasses any suitable medical device configured to allow an operator to access and view internal body anatomy of a subject (e.g., patient) and/or to deliver medical instruments or accessory devices, such as, for example, tomes, biopsy forceps, graspers, baskets, snares, probes, scissors, retrieval devices, lasers, and/or other tools, into the subject’s body. The medical devices herein may be inserted into a variety of body lumens and/or cavities, such as, for example, the urinary tract or gastrointestinal tract. It will be appreciated that, unless otherwise specified, endoscopes, bronchoscopes, choledochoscope, gastroscopes, endoscopic ultrasonography (“EUS”) scopes, colonoscopes, ureteroscopes, laparoscopes, cystoscopes, aspiration scopes, sheaths, catheters, or any other suitable delivery device or medical device may be used in connection with the features described herein.

At least some of the medical devices and systems discussed herein may be used in a variety of medical procedures which require navigating one or more accessory tools through ductal, luminal, or body lumen anatomies. The disclosed medical devices and systems may be inserted via different access points and approaches, e.g., percutaneously, endoscopically, laparoscopically, or some combination thereof.

During endoscopic procedures, accessory devices may be used along with scope devices. Each of the accessory device(s) and the scope device(s) may have one or more actuators, buttons, or the like to be activated by an operator. Accessory devices may be used with a scope device to perform endoscopic retrograde cholangiopancreatography (ERCP) procedures and/or other suitable procedures. ERCP may be used to diagnose and treat conditions of the common bile duct, including, for example, gallstones, inflammatory strictures, leaks (e.g., from trauma, surgery, etc.), and cancer. In an ERCP procedure, a physician may view, through an endoscope, the inside of the stomach and/or the duodenum.

In an ERCP procedure and/or other procedures in which accessory devices are used with a scope device, a guidewire may be delivered to a target location and a shaft of the scope device may be advanced over the guidewire and the guidewire may extend out of an access port at a handle of the scope device. A shaft of the accessory device may then be advanced over the guidewire and through the shaft of the scope device to the target location.

The shaft of the accessory device may be loaded on the guidewire in any suitable manner. In some examples, a long-wire exchange or over-the-wire exchange technique may be used to load the shaft of the accessory device onto the guidewire and to the target location, and in other examples, a rapid exchange technique may be used to load the shaft of the accessory device onto the guidewire and to the target location.

When a long-wire exchange technique is used to load a shaft of an accessory device onto a guidewire, a long guidewire (e.g., approximately 450 centimeters (cm) in length or a guidewire having other suitable lengths) may be utilized and a second person in addition to the medical practitioner holding and operating the scope device may operate the guidewire and load the shaft of the accessory device onto the guidewire by passing the guidewire through a handle or hub of the accessory device. With the long-wire exchange technique, a guidewire channel or lumen of the shaft of the accessory device may be circumferentially closed, which allows for a fully hydrophilic guidewire to be used as liquid may be passed through the guidewire channel or lumen without a risk of fluid leakage through the wall defining the guidewire channel or lumen.

When a rapid exchange technique is used to load a shaft of an accessory device onto a guidewire, a shorter guidewire (e.g., approximately 260 cm in length or a guidewire having other suitable lengths) may be utilized and no second person in addition to the medical practitioner holding and operating the scope device is needed to operate the guidewire and load the shaft of the accessory device onto the guidewire. Rather, the medical practitioner holding and operating the scope device may load the shaft of the accessory device onto the guidewire at a location proximate an accessory port of the scope device and pass the guidewire through a sidewall port or longitudinally extending slot through the wall defining a guidewire channel or lumen (e.g., a strippable or open C-channel) in the shaft of the accessory device that is distal of the handle of the accessory device. However, because liquid may leak through the sidewall port and/or the longitudinally extending slot in the wall of the shaft defining the guidewire channel or lumen if liquid is applied to the guidewire channel or lumen, a fully hydrophilic guidewire cannot typically be used when a rapid exchange technique is used to load the shaft of the accessory devices onto the guidewire.

The devices disclosed herein may facilitate the use of a long-wire technique (e.g., over-the-wire technique) and/or a short-wire technique (e.g., a rapid-exchange technique) for loading guidewire through an accessory device and an endoscope. In some examples, the devices herein may facilitate a single operator (e.g., a medical practitioner) holding and operating an endoscope while also loading the guidewire into and/or through the shaft of the accessory device. Additionally or alternatively, a fully hydrophilic guidewire may be utilized as the guidewire channel or lumen distal of an introducer device disclosed herein may be circumferentially closed and allow liquid to pass through the guidewire channel or lumen without the liquid leaking from a side wall of the channel. The disclosed concepts may have other suitable benefits as discussed herein and/or otherwise.

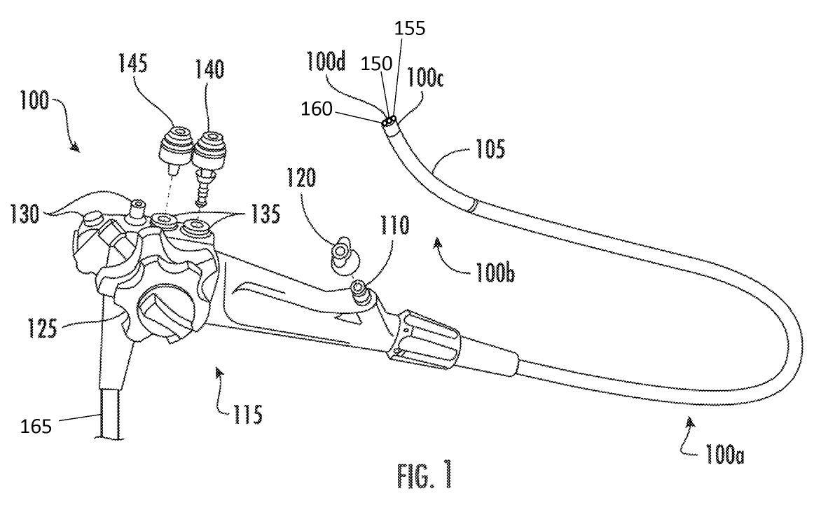

FIG. 1 is a schematic view of an illustrative medical device, such as an endoscope 100 (e.g., a scope device). The endoscope 100 may comprise an elongated shaft 100a that is inserted into a patient. A light source (not explicitly shown) feeds illumination light to a distal portion 100b of the endoscope 100, which may house an imager (e.g., CCD or CMOS imager) (not shown). The light source (e.g., lamp) may be housed in a video processing unit (not explicitly shown) that processes signals that are input from the imager and outputs processed video signals to a video monitor (not explicitly shown) for viewing. The video processing unit may also serve as a component of an air/water feed circuit by housing a pressurizing pump, such as an air feed pump, in the unit.

The endoscope shaft 100a may include a distal tip 100c provided at the distal portion 100b of the shaft 100a and a flexible bending portion 105 proximal to the distal tip 100c. The flexible bending portion 105 may include an articulation joint (not shown) to assist with steering the distal tip 100c. On an end face 100d of the distal tip 100c of the endoscope 100 there may be a gas/lens wash nozzle 150 for supplying gas to insufflate the interior of the patient at the target or treatment area and for supplying water to wash a lens covering the imager. An irrigation opening 155 in the end face 100d may supply irrigation fluid to the treatment area of the patient. Illumination windows (not shown) that convey illumination light to the treatment area and an opening 160 to a working channel extending along the shaft 100a for passing tools to the treatment area may also be included on the face 100d of the distal tip 100c. The working channel may extend along the shaft 100a from the distal opening 160 to a proximal channel opening at an access port 110 positioned at or distal to an operating handle 115 of the endoscope 100. A biopsy valve or cap 120 may be utilized to seal the access port 110 against unwanted fluid egress.

A proximal end region of the operating handle 115 may be provided with knobs 125 for providing remote 4-way steering of the distal tip 100c via wires connected to the articulation joint in the bendable flexible portion 105 (e.g., one knob may control up-down steering and another knob may control for left-right steering). A plurality of video switches 130 for remotely operating the video processing unit may be arranged on a proximal end side of the handle 115. In addition, the handle 115 may be provided with dual valve wells 135. One of the valve wells 135 may receive a gas/water valve 140 for operating an insufflating gas and lens water feed operation. A gas supply line and a lens wash supply line (not explicitly shown) may run distally from the gas/water valve 140 along the shaft 100a and converge at the distal tip 100c proximal to the gas/wash nozzle 150. The other valve well 135 may receive a suction valve 145 for operating a suction operation. A suction supply line runs distally from the suction valve 145 along the shaft 100a to a junction point in fluid communication with the working channel of the endoscope 100.

The operating handle 115 may be electrically and fluidly connected to the video processing unit, via a flexible umbilical 165 and a connector portion extending therebetween. The flexible umbilical 165 may have a gas (e.g., air or CO2) feed line, a lens wash feed line, a suction feed line, an irrigation feed line, a light guide, and an electrical signal cable. The connector portion, when plugged into the video processing unit, may connect the light source in the video processing unit with the light guide. The light guide may run along the umbilical 165 and the length of the endoscope shaft 100a to transmit light to the distal tip 100c of the endoscope 100. The connector portion when plugged into the video processing unit also connects the air pump to the gas feed line in the umbilical 165.

In some configurations, it may be desirable to releasably secure an end effector, an accessory device, or the like to the handle 115 of the endoscope 100. Some illustrative endoscope mounted devices are shown and described in commonly assigned U.S. Patent Application Number 19/066,877, titled MEDICAL DEVICE ACTUATORS AND RELATED METHODS, filed on February 28, 2025, the disclosure of which is incorporated herein by reference for any and all purposes.

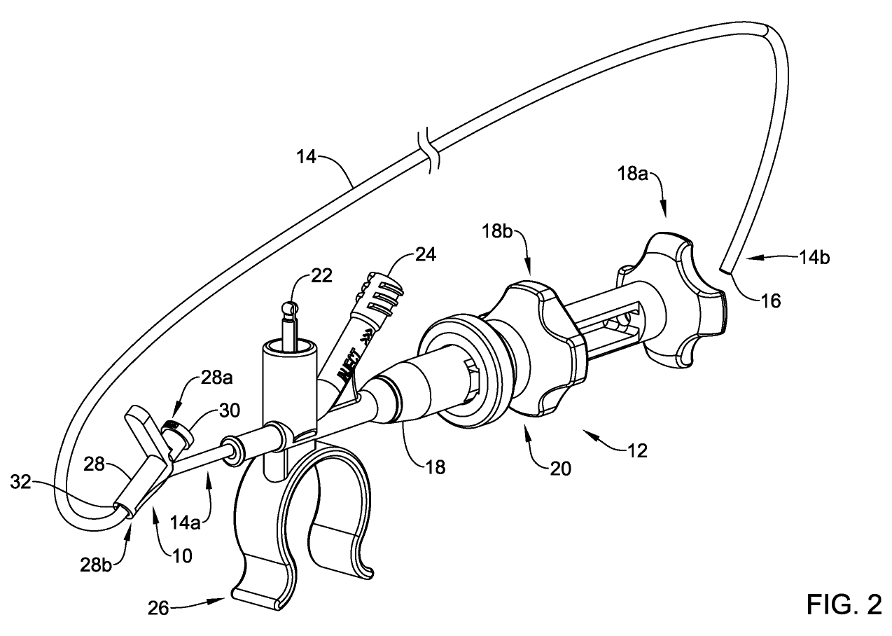

FIG. 2 depicts a schematic perspective view of an accessory device 12 (e.g., a diagnostic device, a treatment device, and/or other suitable medical device) including or otherwise coupled with an introducer 10. The accessory device 12 may be any suitable type of accessory device. In some examples, the accessory device 12 may be configured to facilitate accessing a body lumen. In some examples, the accessory device 12 may be a sphincterotome, but other suitable accessory devices usable with endoscopes 100 are contemplated.

The accessory device 12 depicted in FIG. 2 may include any suitable features or components. For example, the accessory device 12 may include a flexible elongate tube 14 having a proximal end 14a and a distal end 14b. In some examples, the distal end 14b of the elongate tube 14 may have a distal tip 16 and may be configured to be directed toward and/or through an opening of a body lumen. The proximal end 14a of the elongate tube 14 may be connected to, coupled with, or otherwise in communication with a handle 18.

The handle 18 may be any suitable type of handle. In one example, the handle 18 may have a first portion 18a and a second portion 18b. In some examples, the second portion 18b of the handle 18 may be an actuating portion 20 of the handle 18 and a user may adjust the second portion 18b relative to the first portion 18a to control positioning of a distal end of the accessory device 12, as discussed herein or otherwise. Although not required, the handle 18, may be operated with a single hand of a user.

The second portion 18b of the handle 18 may be configured to be adjusted or manipulated to control a position of a distal end 14b of the elongate tube 14 and/or a wire filament (not shown). In some examples, the second portion 18b of the handle 18 may be configured to adjust axially or longitudinally and/or radially or rotationally to adjust a position of the distal end 14b of the elongate tube 14 and/or the wire filament in one or more directions.

When the accessory device 12 includes a wire filament (e.g., a control wire, etc.), the wire filament may be received within a lumen of the elongate tube 14 at least along a proximal portion of the wire filament and may be configured to slide within the lumen. In some examples, the actuating portion 20 of the handle 18 may be coupled to the wire filament and manipulated to slide the wire filament along the elongate tube 14 (e.g., within the lumen of the elongate tube 14). In one example, the actuating portion 20 (e.g., the second portion 18b) of the handle 18 may be longitudinally adjusted relative to the first portion 18a of the handle 18 to apply and remove tension to and/or from the wire filament.

Sliding or adjusting the wire filament may result in the distal end 14b of the elongate tube 14 moving responsively to the sliding of the wire filament. As such, the actuating portion 20 of the handle 18 may be manipulated to control or adjust a position of the distal end 26b of the elongate tube 14, for example, to direct the distal end 14b toward the opening of a body lumen. The distal tip 16 of the elongate tube 14 may be flexible and configured to adjust in response to steering with or adjustment of a control configuration at a proximal end of the accessory device 12 (e.g., a control configuration of or at the handle 18). In some cases, natural tension in or acting on the elongate tube 14 and/or the wire filament may result in the wire filament returning to a relaxed position or state once a tension applied to the wire filament via the handle 18 is released.

The distal tip 16 may be formed from any suitable material configured to bend, deflect, or otherwise move in response to forces acting thereon. In one example, the distal tip 16 may be formed from polytetrafluoroethylene (PTFE), but other suitable materials are contemplated. Accessory devices having a steerable distal tip are described in commonly assigned U.S. Patent Application Publication Number 2023/0372007, titled MEDICAL DEVICE WITH A STEERABLE TIP, and filed on May. 18, 2023, the disclosure of which is hereby incorporated by reference for any and all purposes.

The wire filament, when included with the accessory device 12, may be utilized to electrically cut or remove tissue of the subject (e.g., to cut and cauterize tissue) and may be considered a cautery wire. As such, the handle 18 or other suitable portion of the accessory device 12 may include an electrical connector 22 for an energy source (e.g., a radiofrequency energy source, or the like) to energize the wire filament and facilitate cutting tissue. When the actuating portion 20 of the handle 18 is manipulated to adjust a position of the distal end 14b of the elongate tube 14 and/or utilize the wire filament for cutting tissue, the wire filament may be spaced from the elongate tube 14 and electrified to facilitate cutting tissue at or adjacent to the opening of the body lumen.

A lumen of the one or more lumens of the elongate tube 14 may have a fluid connection at a proximal end for a fluid source. The lumen may be configured to receive and apply a fluid from the fluid source through an opening at the distal end 14b of the elongate tube 14 to a body lumen of the subject. In some examples, the accessory device 12 may include a fluid port 24 in fluid communication with the lumen configured to fluidly couple with the fluid source. The fluid source may be any suitable type of fluid source including, but not limited to, a pressurized fluid source such as a CO₂ tank, a contrast fluid source such as a syringe, tubing, and/or other suitable type of fluid source. In one example, a fluid source may be a syringe with saline, where the syringe may configured to be coupled to the fluid port 24 via a luer lock connection and/or other suitable connection. In some examples, separate lumens and separate fluid ports may be provided for each of a pressurized fluid and saline.

The accessory device 12 may include or may otherwise couple with a mounting component 26 configured to releasably mount the accessory device 12 to the handle 115 of the endoscope 100. In some cases, the mounting component 26 may position the actuating portion 20 of the accessory device 12 on or in close proximity with the endoscope handle 115 allowing a single-user to control both the accessory device 12 and the endoscope 100.

The mounting component 26 may have any suitable configuration. In some examples, the mounting component 26 may be or may include a generally “C”-shaped or omega-shaped bracket configured to be positioned around and form a friction fit or interference fit with the outer surface of the handle 115 of the endoscope 100. Other suitable configurations of the mounting component 26 are contemplated.

The introducer 10 may have any suitable configuration. In some examples, the introducer 10 may be configured to receive the elongate tube 14 and a guidewire extending through the elongate tube 14 of the accessory device 12. In some examples, the introducer 10 may be configured to facilitate removing elongate tube 14 from the guidewire and/or otherwise separating the guidewire and the accessory device 12. In some examples, the introducer 10 may be configured to facilitate delivering the guidewire through the elongate tube 14 and the endoscope 100 to a target location.

Among other features, the introducer 10 may have a housing 28 with a proximal end 28a and a distal end 28b. In some examples, the proximal end 28a of the housing 28 may include a port 30 configured to receive a guidewire and/or couple with a fluid source (e.g., a syringe, tubing, etc.) The distal end 28b of the housing 28 may include an opening 32 (e.g., a distal opening) through which the elongate tube 14 of the accessory device 12 may extend. In some examples, the introducer 10 may include an extension 34 at or between the proximal end 28a and the distal end 28b of the housing 28. Other suitable configurations of the introducer 10 are contemplated including, but not limited to, as discussed herein.

The introducer 10 may be coupled with the accessory device 12 in any suitable manner. In one example, during assembly of the accessory device 12 the proximal end 14a of the elongate tube 14 may be thread through the opening 32 at the distal end 28b of the housing 28 of the introducer 10 and out of the introducer 10. Then, the proximal end 14a of the elongate tube 14 may be coupled with the handle 18 of the accessory device 12. In another example, the introducer 10 may be slid onto a distal end of the elongate tube 14 before or after the elongate tube 14 is coupled with the handle 18 of the accessory device 12. The introducer 10 may be slid in a proximal direction until the introducer 10 reaches a slit, skive, or other suitable opening in the elongate tube 14 through which a tube of the introducer 10 that is configured to receive a guidewire may be inserted into a guidewire lumen of the elongate tube 14. In another example, the housing 28 of the introducer 10 may be formed from a plurality of pieces (e.g., a clam shell configuration, etc.) and before or after coupling the elongate tube 14 with the handle 18 of the accessory device 12, the housing 28 may be coupled together around the elongate tube 14 at a longitudinal location of a slit, skive, or other suitable opening in the elongate tube 14 through which a tube of the introducer 10 configured to receive a guidewire may be inserted into a guidewire lumen of the elongate tube 14. As part of the assembly, the tube configured to receive the guidewire may be inserted into the slit, skive, or other suitable opening. The pieces of the housing 28 may be coupled together with a snap-fit, adhesive, and/or other suitable type of connection. Other suitable configurations for coupling the introducer 10 with the accessory device 12 are contemplated.

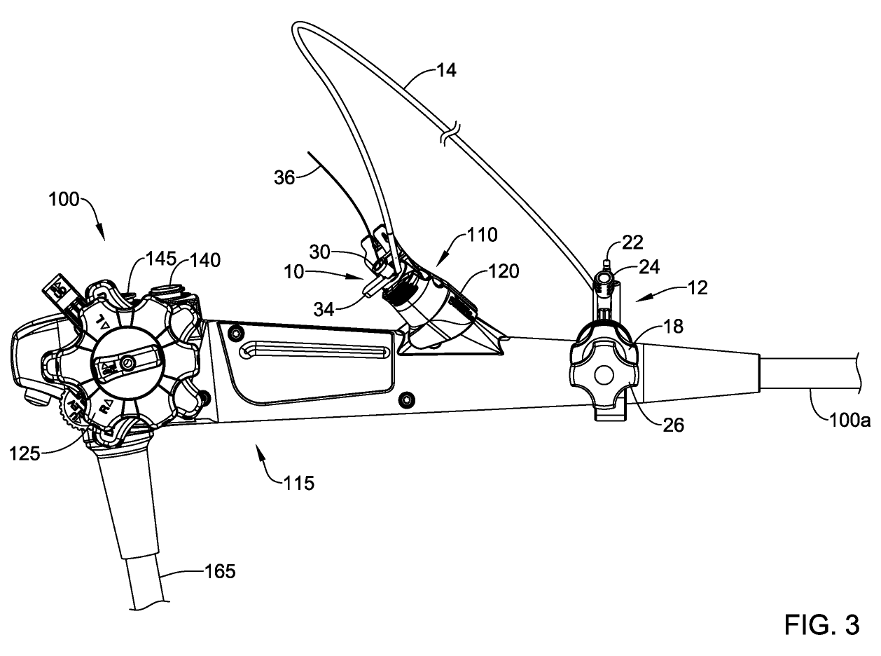

FIG. 3 depicts a schematic side view of an illustrative configuration of a system including the introducer 10 coupled with the accessory device 12 and the accessory device 12 coupled with the endoscope 100. As depicted in FIG. 3, the introducer 10 may be coupled with the elongate tube 14 of the accessory device 12 and positioned at the access port 110 of the endoscope 100. In some examples, the access port 110 of the endoscope 100 may include or may be coupled with an adapter or cap 120 (e.g., a biopsy cap, etc.) and the introducer 10 may be couple with or be inserted into or to the cap 120, but other suitable configurations in which the cap 120 is omitted are contemplated. A guidewire 36 over which the elongate tube 14 travels to a target location may extend through the port 30 of the introducer 10 and out of the access port 110. The accessory device 12 may be coupled with the handle 115 of the endoscope 100 via the mounting component 26. Although the accessory device 12 is depicted in FIG. 3 as being mounted to the handle 115 of the endoscope 100 at a location distal of the access port 110, the accessory device 12 may be coupled to the endoscope 100 at one or more other suitable location and or may not be coupled with the endoscope 100 other than via the elongate tube 14 at the access port 110.

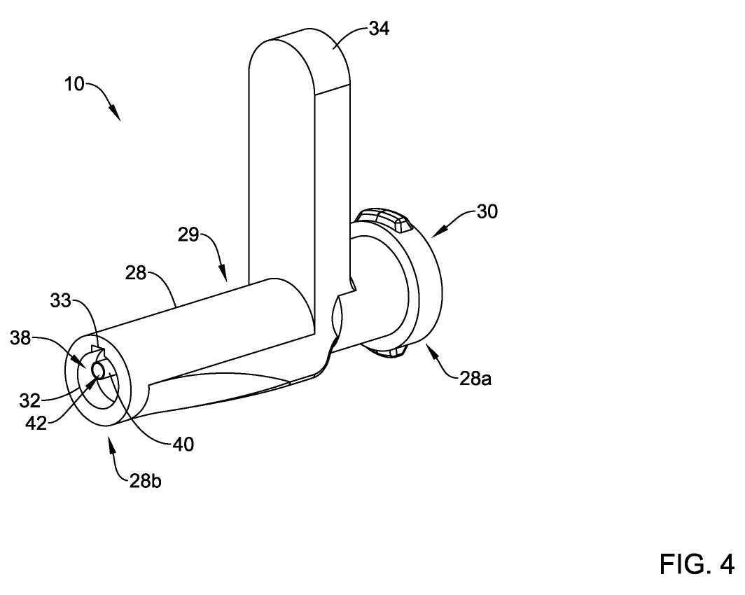

FIG. 4 depicts a schematic perspective view of an illustrative configuration of the introducer 10. As discussed, the introducer 10 may include the housing 28 having the proximal end 28a and the distal end 28b. In some examples, the housing 28 may define the port 30 configured to couple with a fluid source and/or other suitable component. The port 30 may be configured to couple with the fluid source in any suitable manner including, but not limited to, a friction or interference coupling, a bayonet coupling, a threaded coupling, a luer-lock coupling, a Touhy-Borst coupling, and/or in one or more other suitable manners.

The housing 28 may define a single port 30, as depicted for example in FIG. 4, or multiple ports. In some examples, the housing 28 may include a plurality of ports with a first port configured to receive the guidewire (e.g., the first port may be a guidewire port) and a second port configured to receive a fluid from a fluid source, where the first port and the second port may be in fluid communication with a guidewire receiving lumen of the introducer 10. In such an example, the guidewire may be a hydrophilic guidewire and liquid (e.g., water, saline, etc.) may be applied to a guidewire lumen through which the guidewire extends via the second port before or as the guidewire extends through first port to achieve a desired lubricity of the guidewire lumen. In some examples, the housing 28 may include a single port 30. When the housing 28 includes a single port 30, the guidewire receiving lumen of the introducer must be we wetted prior to receiving the guidewire through the port 30 such that a fluid source may couple with the port 30 via a fluid-tight seal (e.g., without interference from the guidewire extending therethrough). Alternatively or additionally, a fluid source coupling may be configured to receive the guidewire while maintaining a fluid-tight seal with the port 30, which may facilitate simultaneously receiving the guidewire at the port 30 and receiving liquid at the port 30. In some examples, when the housing 28 includes a single port 30, a multi-port adaptor (e.g., Y-port adaptor) may be coupled with the port 30 such that the port 30 may receive the guidewire while simultaneously receiving liquid to wet the guidewire lumen(s) through which the guidewire may extend.

The housing 28 may define the opening 32 at the distal end 28b of the introducer 10. The opening 32 may lead to a first lumen 38 defined by the housing 28. In some examples, the opening 32 and the first lumen 38 extending from the opening 32 may be configured to receive the elongate tube 14 of the accessory device 12 and/or a guidewire over which the elongate tube 14 is configured to extend.

The opening 32 may have any suitable shape or configuration designed to receive the elongate tube 14. In some examples, the opening 32 may be circular or generally circular. As depicted in FIG. 4, the opening 32 may be generally circular and include a notch 33. In some examples, the notch 33 may facilitate receiving a blade of the introducer 10 during assembly of the introducer 10 and/or may be utilized for other purposes.

As discussed, the introducer 10 may include an extension 34 that may be configured to facilitate a user (e.g., a medical practitioner) advancing the introducer in a distal direction over the elongate tube 14 with the elongate tube 14 extending through the introducer 10. In some examples, the extension 34 may be located at a longitudinal location of the introducer 10 configured to prevent the introducer 10 from being inserted into the access port 110 or adaptor or cap 120 beyond a desired distance to prevent the introducer 10 from becoming stuck in the endoscope 100. In some examples and as depicted in FIG. 4, the extension 34 may extend laterally outward from a body portion 29 of the housing 28, but other suitable configurations of the extension 34 are contemplated. In some examples and as depicted in FIG. 4, the extension 34 may be positioned at a location along the housing 28 between and spaced from the proximal end 28a and the distal end 28b, but it is contemplated that the extension 34 may be located at the proximal end 28a and/or the distal end 28b of the housing 28. The lateral extension and/or the longitudinal location of the extension 34 may facilitate advancing the introducer 10 over the elongate tube 14 by providing leverage for applying a force in a distal direction to the introducer 10 and by providing a surface that may engage the access port 110 or the adaptor or cap 120 to prevent further advancement of the introducer 10 in a distal direction.

The housing 28 of the introducer 10 may be a single component or multiple components coupled together. In one example and as depicted in FIG. 4, the housing 28 may be a single, monolithic component. In some examples, the housing 28 may be formed from multiple components coupled with one another. In one example, the housing 28 formed from multiple components having a clam shell configuration in which two or more components are hingedly coupled and may also snap or clasp together. Other suitable configurations of the housing 28 are contemplated.

The housing 28 of the introducer 10 may be formed in any suitable manner. For example, the housing 28 may be formed using a molding technique, a machining technique, an over molding technique, a heat staking technique, adhesive coupling of components, snap connection of components, interference coupling of components, and/or other suitable forming and/or coupling techniques.

The housing 28 may be formed from any suitable material. Example suitable materials for forming the housing 28 include, but are not limited to, metals, polymers, plastics, acrylonitrile butadiene styrene (ABS), polycarbonate (PC), and/or other suitable materials. In one example, the housing 28 may be formed from one or more plastic materials.

As depicted in FIG. 4, the introducer 10 may include a tube 40 configured to receive a guidewire extending through the introducer 10. The tube 40 may define a lumen 42 configured to receive the guidewire. The tube 40 may extend from a proximal end at or in fluid communication with the port 30 of the introducer 10 to a distal end. The distal end of the tube 40 may be located in the first lumen 38 and proximal of the opening 32, may be located at the opening 32, or may be located distal of the opening 32. In some examples, the tube 40 may be aligned (e.g., radially aligned) with the notch 33, but other suitable configurations are contemplated.

The tube 40 of the introducer 10 may have any suitable configuration. For example, the tube 40 may be a hypotube, an extruded tube, a molded tube, a machined tube, a rolled and welded tube, and/or may have one or more other suitable configurations. In one example, the tube 40 may be a hypotube, but other suitable configurations of the tube 40 are contemplated. In some examples, the tube 40 may have a smooth distal end. In one example, the distal end of the tube 40 and/or other portions of the tube 40 may be tumbled smooth, ground smooth, and/or otherwise configured to be smooth to avoid sharp edges that may negatively impact one or more coatings on a guidewire extending through the tube 40.

The tube 40 may be formed from any suitable material. Example suitable materials for forming the housing 28 include, but are not limited to, metals, polymers, plastics, a rigid material, a flexible material, 304 stainless steel, coatings, plastic coatings, and/or other suitable materials. In one example, the tube 40 may be formed from one or more rigid, metal materials. In one example, the tube 40 may be formed from plastic material. In one example, the tube 40 may be formed from a metal material dipped in plastic to have an exterior surface formed of plastic. In one example, the tube 40 may have an inner surface formed with (e.g., lined with) plastic material.

The tube 40 may be coupled with the housing 28 in any suitable manner. For example, the tube 40 may be inserted into the housing 28 and secured in place with a friction or interference fit and/or adhesive, the housing 28 may be over molded onto the tube 40, components of the housing 28 may be coupled together over the tube 40 using adhesive and/or other suitable connection techniques, the housing 28 may be heat-staked about the tube 40, and/or the tube 40 may be coupled with the housing 28 in one or more other suitable manners. In one example, the housing 28 may be formed from a polymer and over molded onto the tube 40 to form the introducer 10, but other suitable configurations for forming the introducer 10 with the tube 40 and the housing 28 are contemplated.

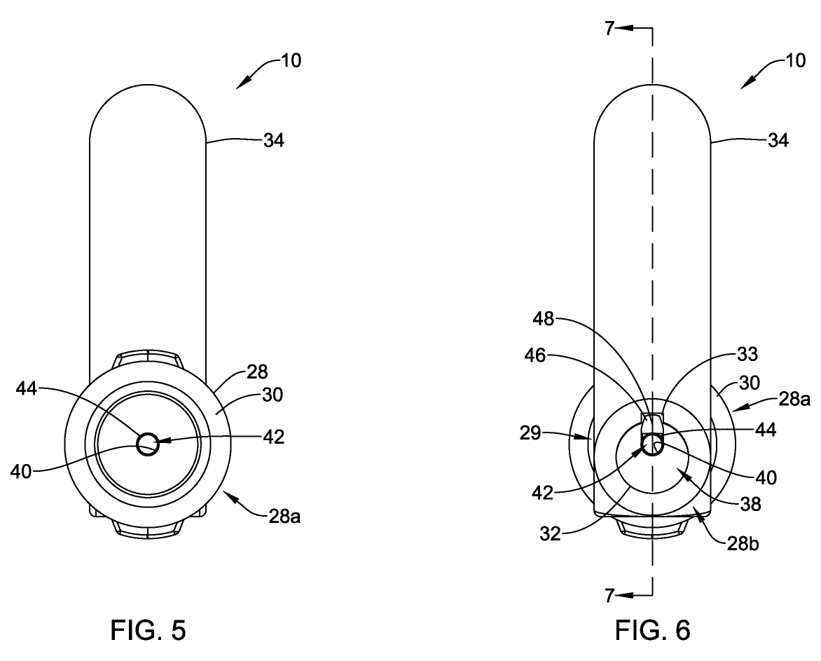

FIG. 5 depicts a schematic proximal end view of the introducer 10. As depicted in FIG. 5, the housing 28 may define the port 30 at the proximal end 28a of the housing 28 and the tube 40 extending through a second lumen 44 of the housing 28 may be in fluid communication with the port 30. In some examples, the second lumen 44 may extend from the port 30 to the first lumen 38. The tube 40 may extend into the port 30 or may have a proximal end that terminates within the second lumen 44 distal of the port 30. Other suitable configurations of the port 30 relative to the tube 40 are contemplated.

FIG. 6 depicts a schematic distal end view of the introducer 10. As depicted in FIG. 6, the housing 28 may define the first lumen 38 extending from the opening 32 at the distal end 28b of the housing 28 and the second lumen 44 of the housing 28 configured to receive the tube 40. As discussed, the tube 40 may be radially aligned with the notch 33, but other suitable configurations are contemplated.

As depicted in FIG. 6, the introducer 10 may include a blade 46 located within the first lumen 38 of the housing 28 between the proximal end 28a and the distal end 28b of the housing 28. In some examples, the blade 46 may be configured to cut through a wall of the elongate tube 14 extending through the housing 28. For example, the blade 46 may be positioned at a location in the housing 28 that is configured to align with an outer wall defining a guidewire lumen of the elongate tube 14 when the elongate tube 14 has been inserted into and through the first lumen 38 to facilitate cutting through the wall of the elongate tube 14 and into the guidewire lumen. In some examples, the blade 46 may have a cutting edge 48 facing a distal direction such that the blade 46 cuts through a wall of the elongate tube 14 and into the guidewire lumen of the elongate tube 14 as the introducer 10 is advanced in a distal direction along the elongate tube 14 and/or as the elongate tube 14 is advanced in a proximal direction through the introducer 10.

The blade 46 may be positioned within the housing 28 between an outer surface of the tube 40 and an inner surface of the housing 28 defining the first lumen 38. The blade 46 may extend from the inner surface of the housing 28 defining the first lumen 38, from the tube 40, or from the inner surface of the housing 28 and from the tube 40. As depicted in FIG. 6, the blade 46 may extend from tube 40 toward the inner surface of the housing 28 defining the first lumen 38. In some examples, the tube 40 may be configured to protect a guidewire extending through the lumen 42 of the tube 40 from the blade 46.

The blade 46 may be formed from any suitable material. In some examples, the blade 46 may be formed from a hardenable steel. In some examples, forming the blade 46 from hardenable steel may facilitate forming the edge 48 with a grinding process. Other suitable configurations of the blade 46 are contemplated.

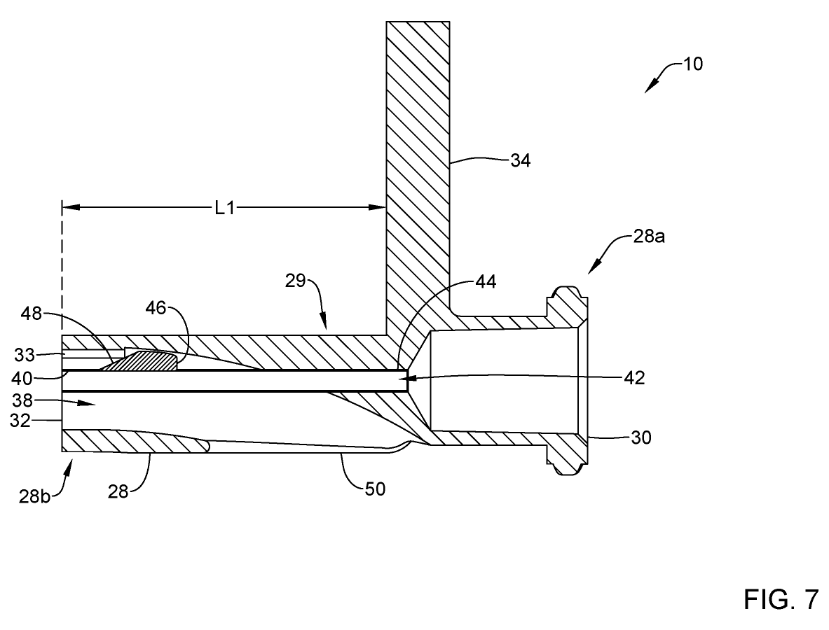

FIG. 7 depicts a schematic cross-section view of the introducer 10. As depicted for example in FIG. 7, the introducer 10 may include the housing 28, the tube 40 configured to receive the guidewire, and the blade 46 configured to engage and cut through a wall of the elongate tube 14. The blade 46 may extend from the tube 40 toward an inner surface of the housing 28, but other suitable configurations are contemplated. The cutting edge 48 of the blade 46 may be facing the opening 32 such that the blade 46 may cut the elongate tube 14 as the introducer 10 is advanced in the distal direction relative to the elongate tube 14 and/or the elongate tube 14 is advanced in the proximal direction relative to the introducer 10.

When the blade 46 extends from the tube 40, the blade 46 and the tube 40 may be formed from a single piece of material or coupled together in one or more manners. In some examples, when the blade 46 and the tube 40 are formed from separate components and are joined together, the blade 46 and the tube 40 may be coupled together with one or more laser welds, solder, and/or other suitable joining techniques.

The tube 40 may define the lumen 42 and extend from the port 30 or a location in fluid communication with the port 30, through the second lumen 44, into the first lumen 38 configured to receive the elongate tube 14, and to the opening 32 at the distal end 28b of the housing 28. The tube 40 may be positioned in the first lumen 38 such that the tube 40 may be aligned with a guidewire lumen of the elongate tube 14 and one or more additional lumens of the elongate tube 14 may be located in the first lumen 38 and spaced from the blade 46. Other suitable configurations of the tube 40 relative to the housing 28 are contemplated.

The first lumen 38 of the housing 28 may extend from the opening 32 (e.g., a first opening or distal opening) at the distal end 28b of the housing 28 to an opening 50 (e.g., a second opening or proximal opening) in the housing 28 proximal of the opening 32. In some examples, the second opening 50 may be in a side wall of the body portion 29 of the housing 28 between the proximal end 28a and the distal end 28b, as depicted for example in FIG. 7, may be at a proximal end of the housing 28, and/or may be at one or more other suitable locations.

In some cases, a portion of the housing 28 forming the introducer 10 may be adapted to be inserted into the access port 110 of the endoscope 100. In some cases, an insertion depth of the housing 28 may be limited by the relative position of the extension 34. The housing 28 may be considered as defining a length L1 that corresponds to a length from a distal end 28b of the housing 28 to where the extension 34 limits insertion depth.

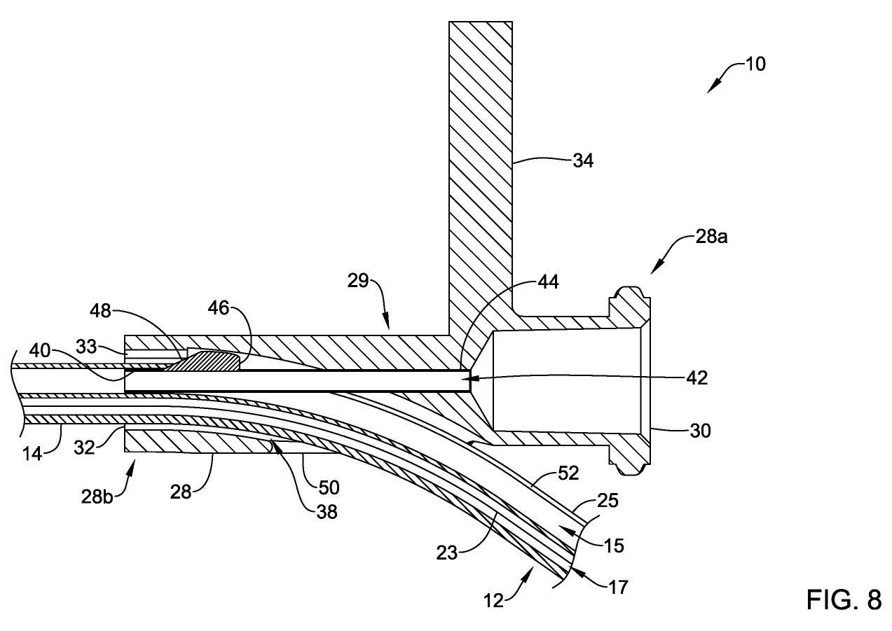

In one example of using the introducer 10, a user may slide the elongate tube 14 having a plurality of lumens and configured to track over a guidewire to a target location into the opening 32 and into the first lumen 38. When inserting the elongate tube 14 into the first lumen 38, a user may align a guidewire lumen of the elongate tube 14 with the tube 40 configured to receive a guidewire and align one or more lumens of the elongate tube 14 with a space within the first lumen 38 below the tube 40, where the blade 46 may be positioned on or proximate a top of the tube 40. FIG. 8 depicts a schematic cross-section view of the introducer 10 with the elongate tube 14 received in the housing 28, as discussed.

As depicted in FIG. 8, the elongate tube 14 may extend through the opening 32 at the distal end 28b of the housing 28, through the first lumen 38, and out of the opening 50 in the sidewall of the housing 28. The tube 40 configured to receive a guidewire may be positioned in or received in a guidewire lumen 15 of the elongate tube 14 and a control wire lumen 17 having a cautery (e.g., electrosurgical) and/or control wire 23 (e.g., a wire filament, etc.) extending therethrough may be positioned in the first lumen 38 below the tube 40. As the elongate tube 14 is inserted into and/or through the first lumen 38 of the housing 28, the blade 46 may extend through an outer wall 25 of the elongate tube 14 defining the guidewire lumen 15 of the elongate tube 14 and the outer wall 25 may engage the cutting edge 48 of the blade 46. As depicted in FIG. 8, as the elongate tube 14 passes the blade 46, the cutting edge 48 of the blade 46 engages the outer wall 25 and cuts the outer wall 25 such that the outer wall 25 defining the guidewire lumen 15 proximal of the cutting edge 48 may have a cut surface 52. For example, as depicted in FIG. 8, the outer wall 25 of the elongate tube 14 distal of the cutting edge 48 has hatching indicating this portion of the outer wall 25 has not been cut by the blade 46 and the outer wall 25 of the elongate tube 14 proximal of the cutting edge 48 does not have hatching indicating this portion of the outer wall 25 has been cut by the blade 46.

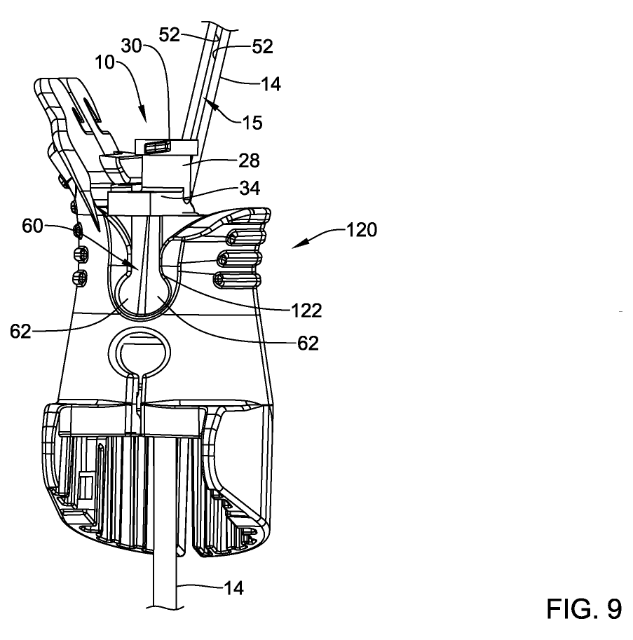

FIG. 9 is a perspective view of the introducer 10 coupled with the adaptor or cap 120. As discussed, the adaptor or cap 120 may be part of or coupled with the access port 110 of the endoscope 100.

As depicted in FIG. 9, the introducer 10 may include one or more connectors 60 configured to facilitate a releasable connection with the cap 120, the access port 110, and/or one or more other suitable components of the endoscope 100 and/or the accessory device 12. In some examples, the connector(s) 60 may be part of the housing 28 of the introducer 10. In some examples, the connector 60 may extend from the extension 34, but other suitable configurations are contemplated.

The connector(s) 60 may be any suitable type of connector including, but not limited to, a clip, a snap connector, a thread, an arm, a leg, a finger, a protrusion, an indent, a mating shape, a flexible component, a resilient component, a biased component, and/or other suitable configuration. The cap 120, the access port 110, and/or one or more other suitable components of the endoscope 100 and/or the accessory device 12 may have one or more connectors 122 configured to receive the connector 60 of the introducer 10. In one example and as depicted in FIG. 9, the connector 60 of the introducer 10 may include a plurality of legs 62 that are biased outward and may be flexed inward (e.g., toward one another) to engage the connector 122, formed as an opening configured to receive the legs 62 of the introducer 10 when the legs 62 are flexed inward and allow for the legs 62 to automatically adjust outward to fill a circumference of and/or mate with the opening forming the connector 122. In some examples, the connection between the connector 60 of the introducer 10 and the connector 122 of the cap 120 may be a snap connection. Other suitable connection types are contemplated for coupling the introducer 10 with or with respect to the endoscope 100 and/or the accessory device 12.

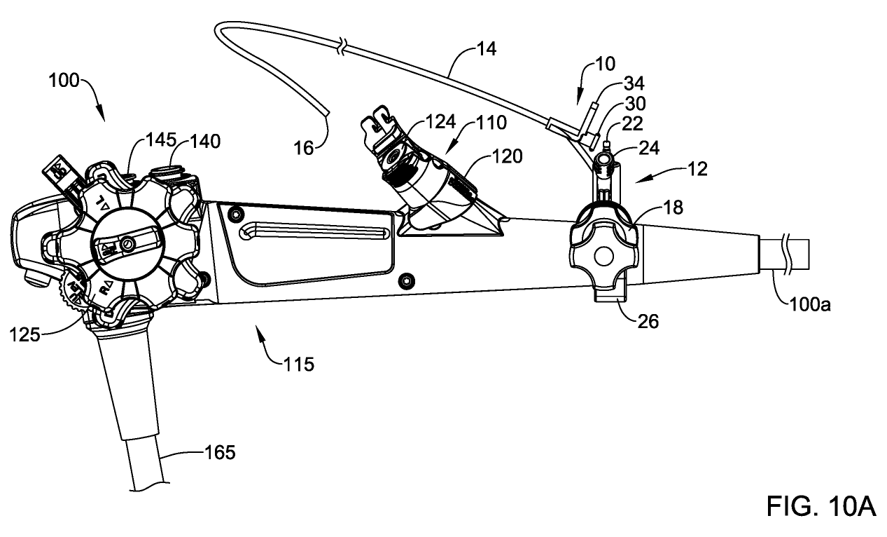

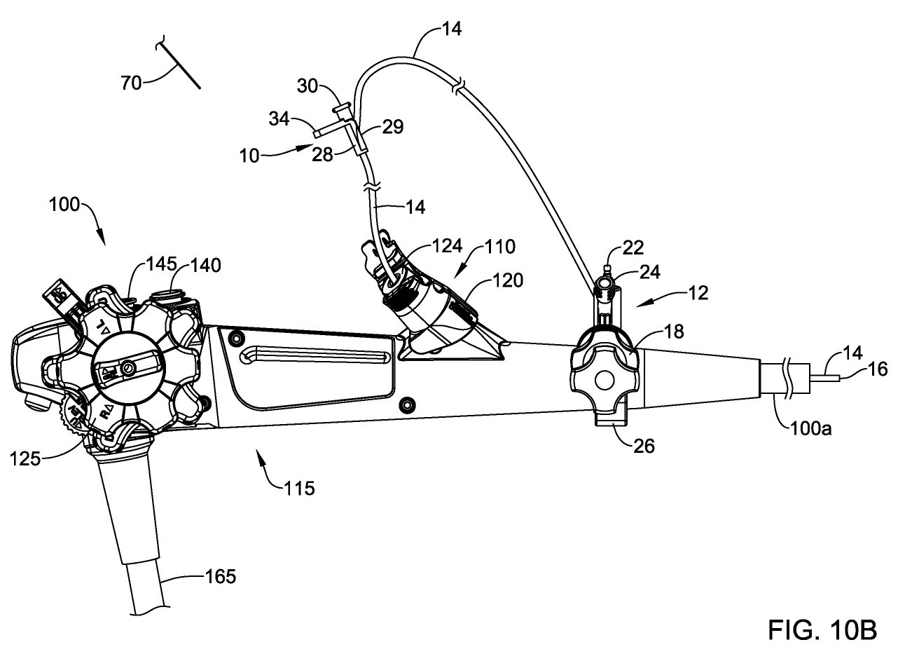

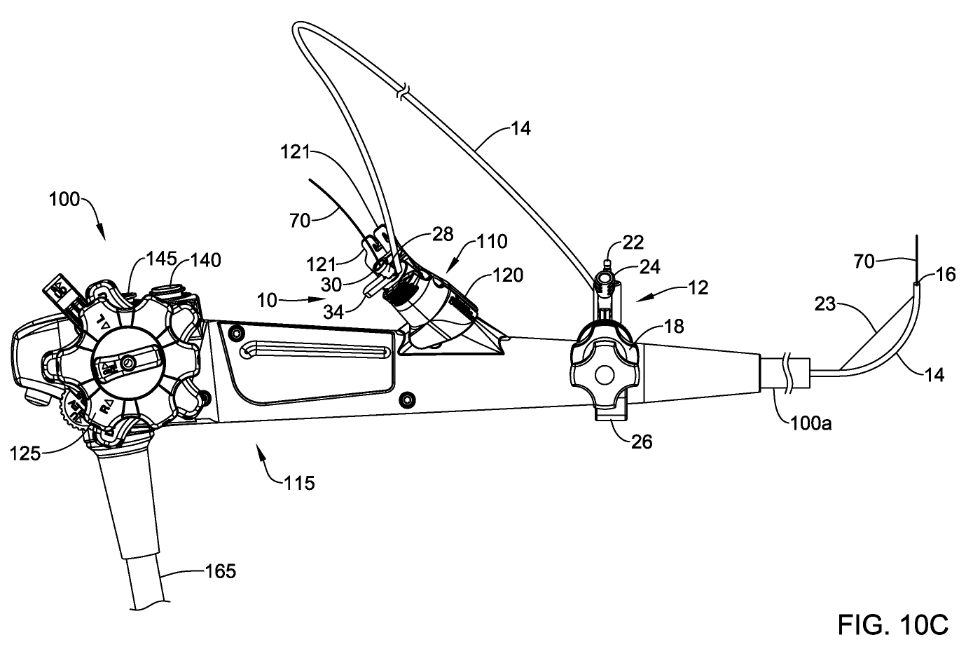

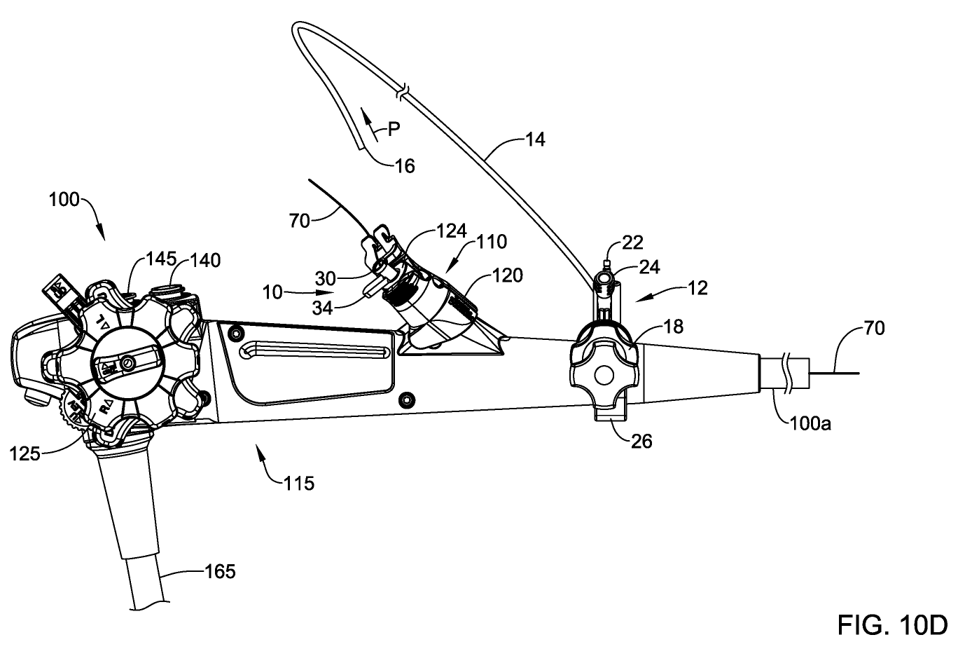

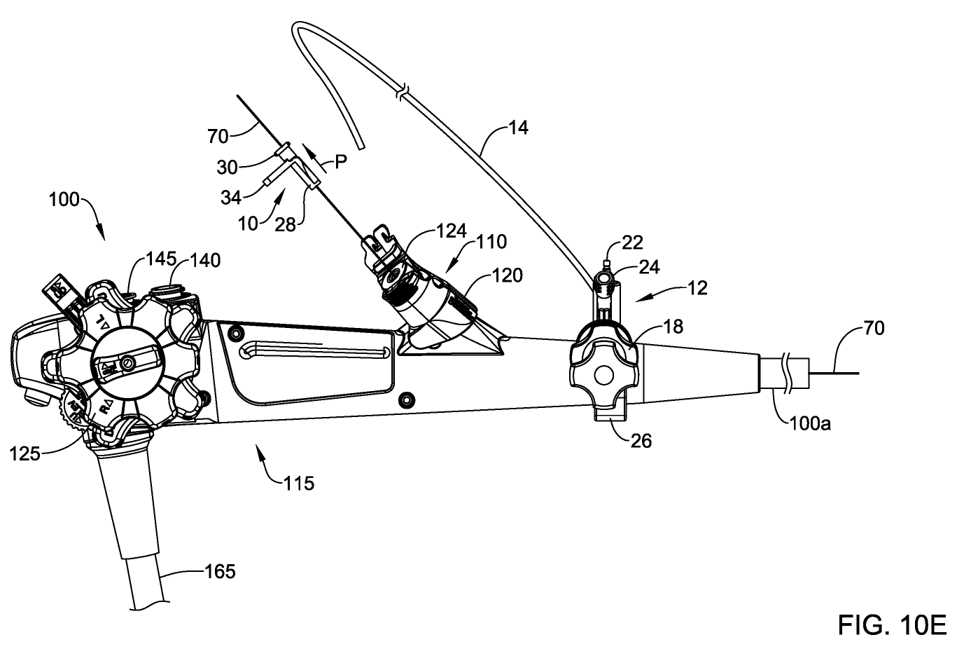

FIGS. 10A-10E depict schematic side views of the system comprising the endoscope 100, the introducer 10, and the accessory device 12 in steps of an illustrative method of using the system. Although the steps of the method are depicted and described with respect to the endoscope 100, the introducer 10, and the accessory device 12 described herein, the method and/or individual steps thereof may be used with other suitable medical systems or system components.

FIG. 10A depicts a schematic side view of the accessory device 12 coupled with the endoscope 100 and the introducer 10 positioned at or proximate a proximal end of the elongate tube 14. When the introducer 10 is coupled with the elongate tube 14 of the accessory device 12, the tube 40 of the introducer 10 may be positioned in the guidewire lumen 15 of the elongate tube 14 and the blade 46 may be aligned with and/or engaged with the outer wall 25 defining the guidewire lumen 15, for example as depicted in FIG. 8. Although the accessory device 12 is coupled with the operating handle 115 of the endoscope 100 via the mounting component 26, the distal tip 16 of the elongate tube 14 has not yet been inserted into the endoscope 100.

FIG. 10B depicts a schematic side view of the system depicted in FIG. 10A, with the elongate tube 140 of the accessory device 12 inserted into the access port 110 via the adaptor or cap 120 and the introducer 10 advanced in a distal direction along the elongate tube 14. In some examples, the introducer 10 may be advanced over the elongate tube 14 by a user applying a force in the distal direction on the extension 34 and/or the body portion 29 of the housing 28 to slide the introducer 10 along the elongate tube 14. Additionally or alternatively, the user may hold the introducer 10 and advance the elongate tube 14 in a proximal direction relative to the introducer to advance the introducer in the distal direction relative to the elongate tube 14. Once the elongate tube 14 is inserted through the endoscope 100 a desired distance, the guidewire 70 may be inserted into the introducer and passed through the guidewire lumen of the elongate tube to a target location.

Advancing the introducer 10 in the distal direction relative to the elongate tube 14 may result in cutting a wall defining a guidewire lumen (e.g., the outer wall 25 defining the guidewire lumen 15, as discussed herein) extending through the elongate tube. A portion of the elongate tube 14 proximal of the introducer 10 may have a longitudinally extending slit or slot extending from the guidewire lumen 15 to the exterior of the elongate tube 14 and the guidewire lumen 15 of a portion of the elongate tube 14 distal of the introducer 10 may be circumferentially enclosed.

FIG. 10C depicts a schematic side view of the system depicted in FIGS. 10A and 10B, with the introducer 10 positioned at or proximate and/or coupled with the access port 110 and/or the adaptor or cap 120 and the elongate tube 14 advanced in a distal direction D, through the operating handle 115 and the shaft 100a of the endoscope 100, to a location of or proximate a target location. In some examples, a distal portion of the elongate tube 14 may be bent or curved, as depicted for example in FIG. 10C, by tensioning the control wire 23 (e.g., by adjusting the handle 18 to apply a proximal force to the control wire 23) and/or in one or more other suitable manners. When the accessory device 12 is an electrosurgical tool, electrical power may be provided to the control wire 23 via the electrical connector 22 to facilitate cutting and/or cauterizing the tissue at or proximate the body lumen.

As depicted in FIG. 10C, a guidewire 70 has been inserted into and through the introducer 10, the elongate tube 14, and the adaptor or cap 120 of or coupled with the access port 110 of the endoscope 100. In some examples, the guidewire 70 may be inserted into a body lumen of a patient to locate a distal portion of the guidewire 70 at or proximate a target location. A proximal end of the guidewire 70 may pass through the endoscope 100 and exit the endoscope 100 at the access port 110 and/or the adaptor or cap 120 and extend through the introducer 10 and out of the port 30.

Prior to or while advancing the guidewire 70 through the elongate tube 14, a syringe or other fluid source may be coupled with the port 30 of the introducer 10. With the syringe or other fluid source coupled with the port 30, a fluid from the syringe or other fluid source may be pumped into the guidewire lumen of the elongate tube 14, through the port 30, and through the tube of the introducer 10 extending into the guidewire lumen of the elongate tube 14. As the portion of the elongate tube 14 distal of the introducer 10 is circumferentially closed, the liquid may be provided to the guidewire lumen without risk of leaking the fluid through a wall defining the guidewire lumen and a fully hydrophilic guidewire may be used as the guidewire 70.

As depicted in FIG. 10C, the introducer 10 may extend into and/or may couple with the access port 110 and/or the adaptor or cap 120. In some examples, the housing 28 of the introducer 10 may extend through one or more seals of the access port 110 and/or the adaptor or cap 120. With the introducer 10 extending through the seal(s) of the access port 110 and/or the adaptor or cap 120 rather than the guidewire 70 extending directly through the seal(s), a user may have more tactile feel or feedback as the guidewire 70 is inserted into, adjusted in, and/or positioned within a body lumen.

A proximal portion of the guidewire 70 extending proximal of the introducer 10 may be threaded through and/or otherwise coupled with one or more guidewire holds 121. The one or more guidewire holds 121 may be configured to receive and create a friction fit coupling with the guidewire 70 to longitudinally hold the guidewire 70 at a desired location.

FIG. 10D depicts a schematic side view of the system depicted in FIGS. 10A-10C, with the introducer 10 positioned at or proximate and/or coupled with the access port 110 and/or the adaptor or cap 120 and the elongate tube 14 advanced in a proximal direction P and separated from the guidewire 70 and the endoscope 100. In some examples, the elongate tube 14 of the accessory device 12 may be removed from the endoscope 100 and the introducer 10 by a user applying a force in the proximal direction P on the elongate tube 14 while holding the introducer 10 and/or the adaptor or cap 120 in place to advance the elongate tube 14 in the proximal direction P over the guidewire 70 until the distal tip 16 has passed through the introducer 10 and is separated from the guidewire 70.

As the elongate tube 14 is advanced in the proximal direction relative to the introducer 10, the blade 46 of the introducer 10 may cut the outer wall defining the guidewire lumen of a portion of the elongate tube 14 that was distal of the introducer 10 prior to the proximal advancement of the elongate tube 14. In some examples, the outer wall defining the guidewire lumen may be cut through the distal tip 16. Alternatively or additionally, a material of the elongate tube 14 may change configuration (e.g., material type, thickness, density, durometer, etc.) along the length of the elongate tube 14 and the outer wall defining the guidewire lumen may become more difficult to cut near the distal tip 16, which may signal to a user that the user should stop advancing the elongate tube 14 in the proximal direction P so as not to cut through the distal tip 16 of the elongate tube 14.

FIG. 10E depicts a schematic side view of the system depicted in FIGS. 10A-10D, with the introducer 10 advanced in the proximal direction P over the guidewire 70. Although the introducer 10 is depicted in FIG. 10E with the guidewire 70 extending through the introducer 10, the introducer 10 may be fully removed from the guidewire 70. To facilitate removing the introducer 10 from the guidewire 70, the guidewire 70 may be disengaged or uncoupled from the guidewire holds 121 of the access port 110 and/or the adaptor or cap 120. With the guidewire 70 separated from the guidewire holds 121, the user may apply a proximal force to the extension 34 and/or the housing 28 in the proximal direction P to remove the introducer 10 from a seal(s) 124 of or at the access port 110 and/or the adaptor or cap 120 and slide the introducer along the guidewire 70.

With the introducer 10 removed from the endoscope 100 and the guidewire 70, one or more additional accessory devices may be used with the endoscope 100 by advancing the additional accessory device over the guidewire 70. When all procedures with the endoscope 100 are complete, the guidewire 70 and/or the shaft 100a of the endoscope 100 may be removed from the body lumen.

In some examples, it may be desirable to ensure the introducer 10 does not slide past a longitudinal location of the elongate tube 14 and/or the elongate tube 14 is not pulled proximally past the longitudinal location of the elongate tube 14. Preserving a portion of the elongate tube 14 may allow the accessory device 12 to be reused. In one example, an inner diameter of the guidewire lumen of the elongate tube 14 may have a reduced inner diameter at or proximate a location at which relative movement of the introducer 10 and the elongate tube 14 is to stop such that a user can feel advancement of the introducer 10 along the elongate tube 14 become more difficult, which signals to a user to stop the relative movement. In one example, the elongate tube 14 may include one or more visual indicators and/or tactile indicators that inform the user that the relative movement of the introducer 10 and the elongate tube 14 should stop so as to be able to reuse the accessory device 12 for another step of the procedure as would be possible with standard rapid exchange devices.

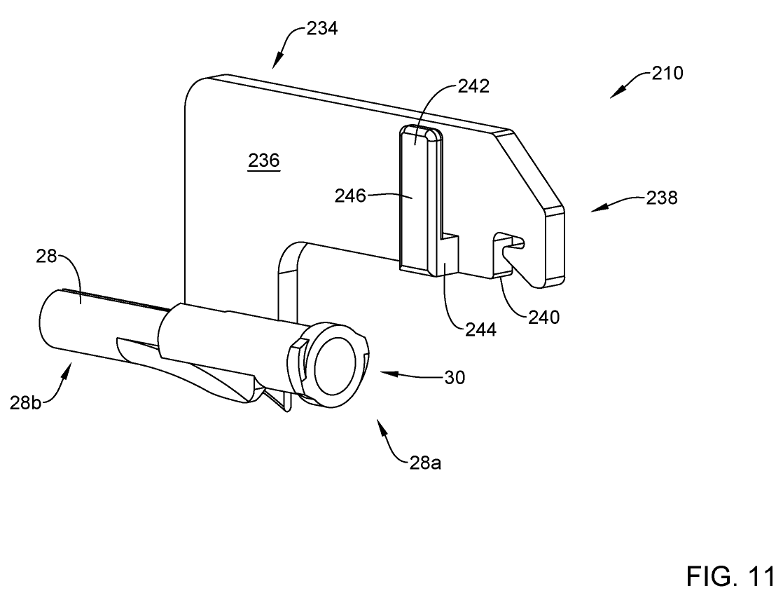

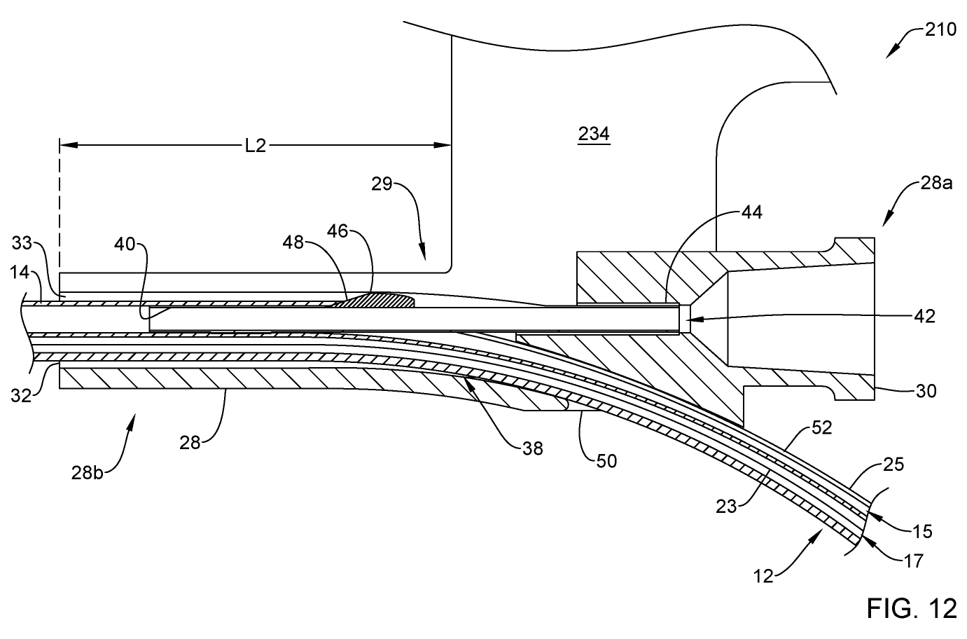

In some instances, the introducer 10 may be configured to facilitate securing the guidewire 70 relative to the introducer 10. FIG. 11 depicts a schematic perspective view of an illustrative introducer 210 that may be used in place of the introducer 10. FIG. 12 depicts a schematic cross-sectional view of the introducer 210 with the elongate tube 14 received in the housing 28. In some cases, as will be discussed, the introducer 210 may be internally the same or similar to the introducer 10, but may be configured to help secure the guidewire 70. The introducer 210 may have any suitable configuration. In some examples, the introducer 210 may be configured to receive the elongate tube 14 and a guidewire extending through the elongate tube 14 of the accessory device 12. In some examples, the introducer 210 may be configured to facilitate removing elongate tube 14 from the guidewire and/or otherwise separating the guidewire and the accessory device 12. In some examples, the introducer 210 may be configured to facilitate delivering the guidewire through the elongate tube 14 and the endoscope 100 to a target location.

Among other features, the introducer 210 may have a housing 28 with a proximal end 28a and a distal end 28b. In some examples, the proximal end 28a of the housing 28 may include a port 30 configured to receive a guidewire and/or couple with a fluid source (e.g., a syringe, tubing, etc.) The distal end 28b of the housing 28 may include an opening 32 (e.g., a distal opening) through which the elongate tube 14 of the accessory device 12 may extend.

The housing 28 may define a single port 30, as depicted for example in FIG. 11, or multiple ports. In some examples, the housing 28 may include a plurality of ports with a first port configured to receive the guidewire (e.g., the first port may be a guidewire port) and a second port configured to receive a fluid from a fluid source, where the first port and the second port may be in fluid communication with a guidewire receiving lumen of the introducer 210. In such an example, the guidewire may be a hydrophilic guidewire and liquid (e.g., water, saline, etc.) may be applied to a guidewire lumen through which the guidewire extends via the second port before or as the guidewire extends through first port to achieve a desired lubricity of the guidewire lumen. In some examples, the housing 28 may include a single port 30. When the housing 28 includes a single port 30, the guidewire receiving lumen of the introducer 210 must be wetted prior to receiving the guidewire through the port 30 such that a fluid source may couple with the port 30 via a fluid-tight seal (e.g., without interference from the guidewire extending therethrough). Alternatively or additionally, a fluid source coupling may be configured to receive the guidewire while maintaining a fluid-tight seal with the port 30, which may facilitate simultaneously receiving the guidewire at the port 30 and receiving liquid at the port 30. In some examples, when the housing 28 includes a single port 30, a multi-port adaptor (e.g., Y-port adaptor) may be coupled with the port 30 such that the port 30 may receive the guidewire while simultaneously receiving liquid to wet the guidewire lumen(s) through which the guidewire may extend.

The housing 28 of the introducer 210 may be a single component or multiple components coupled together. In one example, the housing 28 may be a single, monolithic component. In some examples, the housing 28 may be formed from multiple components coupled with one another. In one example, the housing 28 formed from multiple components having a clam shell configuration in which two or more components are hingedly coupled and may also snap or clasp together. Other suitable configurations of the housing 28 are contemplated.

The housing 28 of the introducer 210 may be formed in any suitable manner. For example, the housing 28 may be formed using a molding technique, a machining technique, an over molding technique, a heat staking technique, adhesive coupling of components, snap connection of components, interference coupling of components, and/or other suitable forming and/or coupling techniques.

The housing 28 may be formed from any suitable material. Example suitable materials for forming the housing 28 include, but are not limited to, metals, polymers, plastics, acrylonitrile butadiene styrene (ABS), polycarbonate (PC), and/or other suitable materials. In one example, the housing 28 may be formed from one or more plastic materials.

The introducer 210 may be coupled with the accessory device 12 in any suitable manner. In one example, during assembly of the accessory device 12 the proximal end 14a of the elongate tube 14 may be thread through an opening 32 at the distal end 28b of the housing 28 of the introducer 210 and out of the introducer 210. Then, the proximal end 14a of the elongate tube 14 may be coupled with the handle 18 of the accessory device 12. In another example, the introducer 210 may be slid onto a distal end of the elongate tube 14 before or after the elongate tube 14 is coupled with the handle 18 of the accessory device 12. The introducer 210 may be slid in a proximal direction until the introducer 210 reaches a slit, skive, or other suitable opening in the elongate tube 14 through which a tube of the introducer 210 that is configured to receive a guidewire may be inserted into a guidewire lumen of the elongate tube 14. In another example, the housing 28 of the introducer 210 may be formed from a plurality of pieces (e.g., a clam shell configuration, etc.) and before or after coupling the elongate tube 14 with the handle 18 of the accessory device 12, the housing 28 may be coupled together around the elongate tube 14 at a longitudinal location of a slit, skive, or other suitable opening in the elongate tube 14 through which a tube of the introducer 210 configured to receive a guidewire may be inserted into a guidewire lumen of the elongate tube 14. As part of the assembly, the tube configured to receive the guidewire may be inserted into the slit, skive, or other suitable opening. The pieces of the housing 28 may be coupled together with a snap-fit, adhesive, and/or other suitable type of connection. Other suitable configurations for coupling the introducer 210 with the accessory device 12 are contemplated.

As noted, the introducer 210 may be configured to accommodate the guidewire 70. In some cases, as shown, the introducer 210 may include an extension 234 at or between the proximal end 28a and the distal end 28b of the housing 28. In some cases, the extension 234 may be integrally molded or otherwise formed as part of the housing 28. In some cases, the extension 234 may be separately formed and subsequently secured to the housing 28. The extension 234 may be adhesively secured to the housing 28, for example, or the extension 234 may include a component (not illustrated) that forms an interference fit within a channel (not illustrated) formed within the housing 28.

In some cases, the extension 234 may be configured to facilitate a user (e.g., a medical practitioner) advancing the introducer 210 in a distal direction over the elongate tube 14 with the elongate tube 14 extending through the introducer 210. In some examples, the extension 234 may be located at a longitudinal location of the introducer 210 configured to prevent the introducer 210 from being inserted into the access port 110 or adaptor or cap 120 beyond a desired distance to prevent the introducer 210 from becoming stuck in the endoscope 100. In some examples and as depicted in FIG. 11, the extension 234 may extend laterally outward from the housing 28, but other suitable configurations of the extension 234 are contemplated. In some examples and as depicted in FIG. 11, the extension 234 may be positioned at a location along the housing 28 between and spaced from the proximal end 28a and the distal end 28b, but it is contemplated that the extension 234 may be located at the proximal end 28a and/or the distal end 28b of the housing 28. The lateral extension and/or the longitudinal location of the extension 234 may facilitate advancing the introducer 210 over the elongate tube 14 by providing leverage for applying a force in a distal direction to the introducer 210 and by providing a surface that may engage the access port 110 or the adaptor or cap 120 to prevent further advancement of the introducer 210 in a distal direction.

The extension 234 may include a body portion 236. A proximal region 238 of the body portion 236 may include a hook 240 that may be used to secure the guidewire 70 relative to the introducer 210. In some cases, the body portion 236 may be dimensioned to locate the hook 240 a greater distance (e.g., in a proximal direction) from the access port 110 than may otherwise be achieved using guidewire hooks that are molded into the cap 120 disposed over the access port 110. In some cases, this may result in the guidewire 70 extending at less of an angle as the guidewire 70 exits the access port 110.

In some cases, the body portion 236 may include a guidewire guide 242. The guidewire guide 242 may have any suitable configuration. In some examples, the guidewire guide 242 may include a base portion 244 that secures or guides the guidewire guide 242 in position on the body portion 236 and an extension portion 246 that extends from the body portion 236 and is spaced from the body portion 236 in order to accommodate the guidewire 70 between the body portion 236 and the extension portion 246. As an example, the guidewire 70 may be tucked into the space between the body portion 236 and the extension portion 246 before extending the guidewire 70 through the hook 240.

The housing 28 may define the opening 32 at the distal end 28b of the introducer 210, as depicted for example in FIG. 12. The opening 32 may lead to a first lumen 38 defined by the housing 28. In some examples, the opening 32 and the first lumen 38 extending from the opening 32 may be configured to receive the elongate tube 14 of the accessory device 12 and/or a guidewire over which the elongate tube 14 is configured to extend.

The opening 32 may have any suitable shape or configuration designed to receive the elongate tube 14. In some examples, the opening 32 may be circular or generally circular. As depicted in FIG. 12, the opening 32 may be generally circular and include a notch 33. In some examples, the notch 33 may facilitate receiving a blade of the introducer 210 during assembly of the introducer 210 and/or may be utilized for other purposes.

As depicted in FIG. 12, the introducer 210 may include a tube 40 configured to receive a guidewire extending through the introducer 210. The tube 40 may define a lumen 42 configured to receive the guidewire. The tube 40 may extend from a proximal end at or in fluid communication with the port 30 of the introducer 210 to a distal end. The distal end of the tube 40 may be located in the first lumen 38 and proximal of the opening 32, may be located at the opening 32, or may be located distal of the opening 32. In some examples, the tube 40 may be aligned (e.g., radially aligned) with the notch 33, but other suitable configurations are contemplated.

In some cases, a portion of the housing 28 forming the introducer 210 may be adapted to be inserted into the access port 110 of the endoscope 100. In some cases, an insertion depth of the housing 28 may be limited by the relative position of the extension 34. The housing 28 may be considered as defining a length L2 that corresponds to a length from a distal end 28b of the housing 28 to where the extension 234 limits insertion depth (e.g., a distal end or distal side of the extension 234), as depicted for example in FIG. 12.

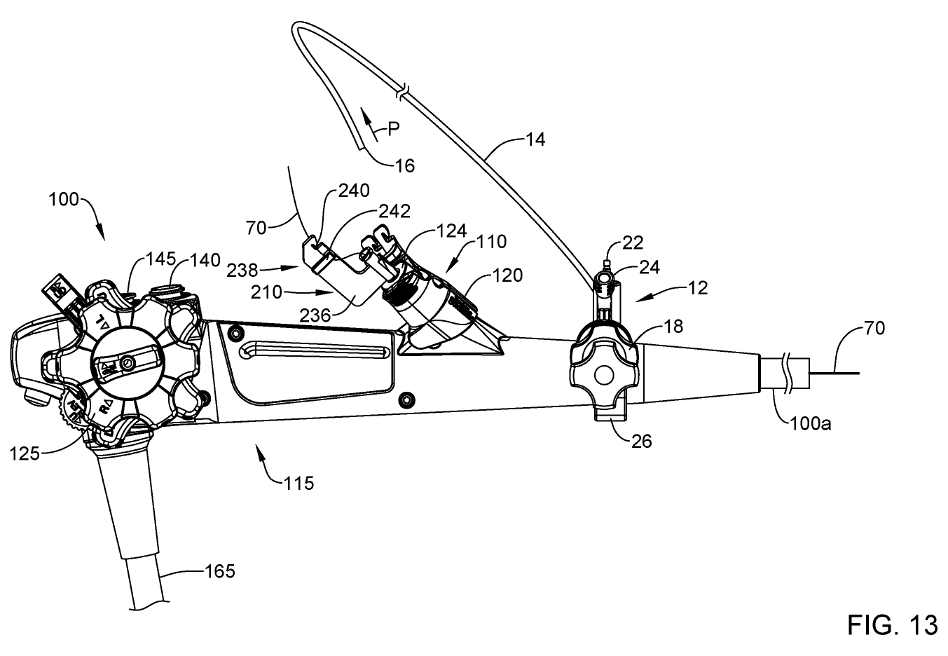

The length L2 may be any suitable length. In some examples, a maximum distance of the length L2 of the introducer 210 may be a distance such that the introducer 210 does not extend beyond a distal end of the seal 124 in the access port 110 and/or the adaptor or cap 120, when the distal side of the extension 234 is in contact with the seal 124, the access port 110, and/or the adaptor or cap 120, as depicted for example in FIG. 13. In some examples, the length L2 of the introducer 210 may be greater than the length L1 of a portion of the introducer 10.

Having L2 be greater than L1 may have one or more benefits. In some examples, having L2 be greater than L1 may facilitate straightening out the tube 40 within the introducer 210 prior to slicing or cutting the tube 40 with the blade 46. Straightening out the tube 40 within the introducer 210 prior to slicing or cutting the tube 40 may facilitate making a reliable and/or consistent cut of the tube 40 and may prevent the tube 40 from popping out of the introducer 210 once sliced or cut.

The tube 40 of the introducer 210 may have any suitable configuration. For example, the tube 40 may be a hypotube, an extruded tube, a molded tube, a machined tube, a rolled and welded tube, and/or may have one or more other suitable configurations. In one example, the tube 40 may be a hypotube, but other suitable configurations of the tube 40 are contemplated. In some examples, the tube 40 may have a smooth distal end. In one example, the distal end of the tube 40 and/or other portions of the tube 40 may be tumbled smooth, ground smooth, and/or otherwise configured to be smooth to avoid sharp edges that may negatively impact one or more coatings on a guidewire extending through the tube 40.

The tube 40 may be formed from any suitable material. Example suitable materials for forming the housing 28 include, but are not limited to, metals, polymers, plastics, a rigid material, a flexible material, 304 stainless steel, coatings, plastic coatings, and/or other suitable materials. In one example, the tube 40 may be formed from one or more rigid, metal materials. In one example, the tube 40 may be formed from plastic material. In one example, the tube 40 may be formed from a metal material dipped in plastic to have an exterior surface formed of plastic. In one example, the tube 40 may have an inner surface formed with (e.g., lined with) plastic material.

The tube 40 may be coupled with the housing 28 in any suitable manner. For example, the tube 40 may be inserted into the housing 28 and secured in place with a friction or interference fit and/or adhesive, the housing 28 may be over molded onto the tube 40, components of the housing 28 may be coupled together over the tube 40 using adhesive and/or other suitable connection techniques, the housing 28 may be heat-staked about the tube 40, and/or the tube 40 may be coupled with the housing 28 in one or more other suitable manners. In one example, the housing 28 may be formed from a polymer and over molded onto the tube 40 to form the introducer 10, but other suitable configurations for forming the introducer 10 with the tube 40 and the housing 28 are contemplated.

As depicted in FIG. 12, the introducer 210 may include the blade 46 located within the first lumen 38 of the housing 28 between the proximal end 28a and the distal end 28b of the housing 28. In some examples, the blade 46 may be configured to cut through a wall of the elongate tube 14 extending through the housing 28. For example, the blade 46 may be positioned at a location in the housing 28 that is configured to align with an outer wall defining a guidewire lumen of the elongate tube 14 when the elongate tube 14 has been inserted into and through the first lumen 38 to facilitate cutting through the wall of the elongate tube 14 and into the guidewire lumen. In some examples, the blade 46 may have a cutting edge 48 facing a distal direction such that the blade 46 cuts through a wall of the elongate tube 14 and into the guidewire lumen of the elongate tube 14 as the introducer 210 is advanced in a distal direction along the elongate tube 14 and/or as the elongate tube 14 is advanced in a proximal direction through the introducer 10.

The blade 46 may be positioned within the housing 28 between an outer surface of the tube 40 and an inner surface of the housing 28 defining the first lumen 38. The blade 46 may extend from the inner surface of the housing 28 defining the first lumen 38, from the tube 40, or from the inner surface of the housing 28 and from the tube 40. As depicted in FIG. 12, the blade 46 may extend from tube 40 toward the inner surface of the housing 28 defining the first lumen 38. In some examples, the tube 40 may be configured to protect a guidewire extending through the lumen 42 of the tube 40 from the blade 46.

The blade 46 may be formed from any suitable material. In some examples, the blade 46 may be formed from a hardenable steel. In some examples, forming the blade 46 from hardenable steel may facilitate forming the edge 48 with a grinding process. Other suitable configurations of the blade 46 are contemplated. The blade 46 may extend from the tube 40 toward an inner surface of the housing 28, but other suitable configurations are contemplated. The cutting edge 48 of the blade 46 may be facing the opening 32 such that the blade 46 may cut the elongate tube 14 as the introducer 210 is advanced in the distal direction relative to the elongate tube 14 and/or the elongate tube 14 is advanced in the proximal direction relative to the introducer 210.

When the blade 46 extends from the tube 40, the blade 46 and the tube 40 may be formed from a single piece of material or coupled together in one or more manners. In some examples, when the blade 46 and the tube 40 are formed from separate components and are joined together, the blade 46 and the tube 40 may be coupled together with one or more laser welds, solder, and/or other suitable joining techniques.

The tube 40 may define the lumen 42 and extend from the port 30 or a location in fluid communication with the port 30, through the second lumen 44, into the first lumen 38 configured to receive the elongate tube 14, and to the opening 32 at the distal end 28b of the housing 28. The tube 40 may be positioned in the first lumen 38 such that the tube 40 may be aligned with a guidewire lumen of the elongate tube 14 and one or more additional lumens of the elongate tube 14 may be located in the first lumen 38 and spaced from the blade 46. Other suitable configurations of the tube 40 relative to the housing 28 are contemplated.

The first lumen 38 of the housing 28 may extend from the opening 32 (e.g., a first opening or distal opening) at the distal end 28b of the housing 28 to an opening 50 (e.g., a second opening or proximal opening) in the housing 28 proximal of the opening 32. In some examples, the second opening 50 may be in a side wall of the body portion 29 of the housing 28 between the proximal end 28a and the distal end 28b, as depicted for example in FIG. 7, may be at a proximal end of the housing 28, and/or may be at one or more other suitable locations.

In one example of using the introducer 210, a user may slide the elongate tube 14 having a plurality of lumens and configured to track over a guidewire to a target location into the opening 32 and into the first lumen 38. When inserting the elongate tube 14 into the first lumen 38, a user may align a guidewire lumen of the elongate tube 14 with the tube 40 configured to receive a guidewire and align one or more lumens of the elongate tube 14 with a space within the first lumen 38 below the tube 40, where the blade 46 may be positioned on or proximate a top of the tube 40.

In some cases, the elongate tube 14 may extend through the opening 32 at the distal end 28b of the housing 28, through the first lumen 38, and out of the opening 50 in the sidewall of the housing 28. The tube 40 configured to receive a guidewire may be positioned in or received in a guidewire lumen 15 of the elongate tube 14 and a control wire lumen 17 having a cautery (e.g., electrosurgical) and/or control wire 23 (e.g., a wire filament, etc.) extending therethrough may be positioned in the first lumen 38 below the tube 40. As the elongate tube 14 is inserted into and/or through the first lumen 38 of the housing 28, the blade 46 may extend through an outer wall 25 of the elongate tube 14 defining the guidewire lumen 15 of the elongate tube 14 and the outer wall 25 may engage the cutting edge 48 of the blade 46. As depicted in FIG. 12, as the elongate tube 14 passes the blade 46, the cutting edge 48 of the blade 46 engages the outer wall 25 and cuts the outer wall 25 such that the outer wall 25 defining the guidewire lumen 15 proximal of the cutting edge 48 may have a cut surface 52. For example, as depicted in FIG. 12, the outer wall 25 of the elongate tube 14 distal of the cutting edge 48 has hatching indicating this portion of the outer wall 25 has not been cut by the blade 46 and the outer wall 25 of the elongate tube 14 proximal of the cutting edge 48 does not have hatching indicating this portion of the outer wall 25 has been cut by the blade 46.

FIG. 13 depicts a schematic side view of the system, with the introducer 210 positioned at or proximate and/or coupled with the access port 110 and/or the adaptor or cap 120 and the elongate tube 14 advanced in a proximal direction P and separated from the guidewire 70 and the endoscope 100. FIG. 13 may be considered as being similar to FIG. 10D, albeit showing the introducer 210 in place of the introducer 10. In some examples, the elongate tube 14 of the accessory device 12 may be removed from the endoscope 100 and the introducer 210 by a user applying a force in the proximal direction P on the elongate tube 14 while holding the introducer 210 and/or the adaptor or cap 120 in place to advance the elongate tube 14 in the proximal direction P over the guidewire 70 until the distal tip 16 has passed through the introducer 210 and is separated from the guidewire 70. In some cases, the guidewire 70 approaches the access port 110 from above. In some cases, the guidewire 70 may be looped through the hook 240 in order to secure the guidewire 70 in position. In some cases, the guidewire 70 may also be looped through the extension portion 246. In some cases, the guidewire 70 may be looped through the hook 240 and the extension portion 246.

As the elongate tube 14 is advanced in the proximal direction relative to the introducer 210, the blade 46 of the introducer 210 may cut the outer wall defining the guidewire lumen of a portion of the elongate tube 14 that was distal of the introducer 210 prior to the proximal advancement of the elongate tube 14. In some examples, the outer wall defining the guidewire lumen may be cut through the distal tip 16. Alternatively or additionally, a material of the elongate tube 14 may change configuration (e.g., material type, thickness, density, durometer, etc.) along the length of the elongate tube 14 and the outer wall defining the guidewire lumen may become more difficult to cut near the distal tip 16, which may signal to a user that the user should stop advancing the elongate tube 14 in the proximal direction P so as not to cut through the distal tip 16 of the elongate tube 14.

The introducer 10, the introducer 210, the accessory device 12, the endoscope 100, the guidewire 70, and/or components thereof may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium- molybdenum alloys (e.g., UNS: R30035 such as MP35-N® and the like), nickel- molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

Some examples of suitable polymers may include, but are not limited to, polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some configurations the sheath can be blended with a liquid crystal polymer (LCP). For example, the mixture can contain up to about 6 percent LCP.

In some configurations, a degree of Magnetic Resonance Imaging (MRI) compatibility is imparted into the introducer 10, the accessory device 12, the endoscope 100, and/or the guidewire 70. For example, the introducer 10, the accessory device 12, the endoscope 100, the guidewire 70, or portions thereof may be made of a material that does not substantially distort the image and create substantial artifacts (i.e., gaps in the image). Certain ferromagnetic materials, for example, may not be suitable because they may create artifacts in an MRI image. The introducer 10, the accessory device 12, the endoscope 100, the guidewire 70, or portions thereof may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example configuration being used in other configurations. The invention's scope is, of course, defined in the language in which the appended claims are expressed.

What is claimed is:

1. A guidewire introducer, comprising:
a housing having a proximal end and a distal end;
a port at the proximal end of the housing;
a tube extending within the housing and in fluid communication with the port;
a lumen extending through the housing; and
a blade positioned within the lumen between the proximal end of the housing and the distal end of the housing.

2. The guidewire introducer of claim 1, wherein the lumen is configured to receive an elongate tube of an accessory device and the tube extends into the lumen.

3. The guidewire introducer of claim 1, wherein the tube is configured to be inserted into a guidewire lumen of an elongate tube of an accessory device extending through the lumen.

4. The guidewire introducer of claim 1, wherein the lumen has a distal opening at the distal end of the housing and a proximal opening through a sidewall of the housing between the distal end of the housing and the proximal end of the housing.

5. The guidewire introducer of claim 1, wherein the tube extends into the lumen and the blade extends between an outer surface of the tube and a surface defining the lumen.

6. The guidewire introducer of claim 1, further comprising:
a tab located between the proximal end and the distal end of the housing.

7. The guidewire introducer of claim 6, wherein tab comprises a guidewire guide.

8. The guidewire introducer of claim 1, wherein the housing is configured to couple with a port of an endoscope.

9. The guidewire introducer of claim 1, wherein the tube is a metallic tube and the blade is metallic.

10. The guidewire introducer of claim 1, wherein the port is a guidewire port and the guidewire introducer further comprises a syringe port.

11. An accessory device for use with an endoscope, the accessory device comprising:
a hub;
an elongate tube having a proximal end at the hub, a distal end, and one or more lumens extending through the elongate tube; and
a guidewire introducer coupled with the elongate tube, the guidewire introducer comprising:
a housing having a proximal end and a distal end;
a tube extending within the housing into a first lumen of the one or more lumens;
a lumen extending through the housing with the elongate tube extending through the lumen; and
a blade extending through a wall of the elongate tube defining the first lumen.

12. The accessory device of claim 11, wherein the tube is positioned in the first lumen and is configured to receive a guidewire extending through the first lumen.

13. The accessory device of claim 12, wherein the elongate tube extends through the distal end of the housing and a sidewall of the housing between the proximal end and the distal end of the housing.

14. The accessory device of claim 12, wherein the guidewire introducer and the elongate tube are configured to adjust longitudinally relative to one another.

15. The accessory device of claim 14, wherein the blade is configured to cut the wall of the elongate tube defining the first lumen as the guidewire introducer and the elongate tube adjust longitudinally relative to one another.

16. A method of using a guidewire introducer with an accessory device for insertion of a guidewire into an endoscope, the method comprising:
cutting a wall defining a guidewire lumen of an elongate tube of the accessory device with a blade of the guidewire introducer as the guidewire introducer is advanced over the elongate tube;
inserting the guidewire through the guidewire introducer and the elongate tube to a target location;
positioning the guidewire introducer proximate a port of the endoscope, the elongate tube extending through the port of the endoscope; and
removing the elongate tube from the endoscope and the guidewire.

17. The method of claim 16, further comprising:
removing the elongate tube from the guidewire introducer,
wherein removing the elongate tube from the guidewire introducer includes cutting the elongate tube with the blade as the elongate tube is adjusted in a proximal direction.

18. The method of claim 16, further comprising:
removing the guidewire introducer from the guidewire.

19. The method of claim 16, further comprising:
extending a housing of the guidewire introducer through a seal at the port of the endoscope.

20. The method of claim 16, further comprising:
coupling a syringe with a port of a housing of the guidewire introducer; and
providing liquid to the guidewire lumen of the elongate tube.
